(19) United States
(12) Patent Application Publication
(10) Pub. No.: US 2020/0188639 A1
(43) Pub. Date: Jun. 18, 2020

(54) VARIABLE STIFFNESS INTRALUMINAL DEVICE

(71) Applicant: KONINKLIJKE PHILIPS N.V., Eindhoven (NL)

(72) Inventors: Jeremy STIGALL, CARLSBAD (CA); Nathan Andrew WILLIAMS, SAN DIEGO, CA (US)

(21) Appl. No.: 16/708,498

(22) Filed: Dec. 10, 2019

Related U.S. Application Data
(60) Provisional application No. 62/780,277, filed on Dec. 16, 2018.

(51) Int. Cl. A61M 25/09; A61M 25/00; A61M 25/01

(52) U.S. Cl. CPC A61M 25/09; A61M 25/0054; A61M 25/0105; A61M 2025/0915; A61M 2025/0002; A61M 2205/123; A61M 2209/045; A61M 2025/09183; A61M 2025/0166

(57) ABSTRACT

In an embodiment, an intraluminal device is disclosed. One embodiment of the intraluminal device comprises a flexible elongate body comprising a distal portion configured to be positioned within a body lumen of a patient and a functional device disposed at the distal portion of the flexible elongate body, the functional device configured to obtain physiological data or perform a treatment within the body lumen. The intraluminal device further comprises a stiffening member moveably disposed within and along a longitudinal length of the flexible elongate body without extending beyond a distal end of the flexible elongate body, the stiffening member being moveable during an intraluminal procedure to selectively vary a stiffness of the intraluminal device.

### CROSS-REFERENCE TO PRIOR APPLICATIONS

This application claims the benefit of U.S. Provisional Patent Application Ser. No. 62/780,277, filed Dec. 16, 2018 which is hereby incorporated by reference herein.

### TECHNICAL FIELD

The present disclosure generally relates to structure of intraluminal devices, such as intravascular catheters and guidewires, that may be used to obtain physiological data (e.g., images, pressure measurements, flow measurements, etc.) associated with a body lumen of a patient. For example, the disclosed intraluminal devices are structured such that the stiffness of the intraluminal devices can be adjusted during a medical procedure.

### BACKGROUND

Cardiovascular diseases (CVD) have been on a steep rise since the last decade. The death toll recorded in 2008 was 17.3 million (about 30% of global deaths) and is estimated to reach 23.3 million by 2030. Heart disease and stroke form the major contributors to total loss due to CVD with 7.3 million and 6.2 million deaths respectively. Developing and under-developed nations of the world bear a greater part (almost 80%) of the total CVD burden. The national census of India 2010-11, recorded a staggering 0.3 million deaths due to diseases of circulatory system which comprised 29.8% of the total deaths. One major cause of CVD is the presence of flow reducing blockages or lesions within blood vessels. For example, accumulation of plaque inside blood vessels can eventually cause occlusion of the blood vessels through the formation of a partial or even a complete blockage. The formation of such blockages can be life-threatening, and surgical intervention is often required to save the lives of afflicted individuals.

Intravascular assessment techniques such as intravascular ultrasound (IVUS) and intravascular pressure measurements can be used to assess a diseased vessel, such as an artery, within the human body to determine the need for treatment, to guide a therapeutic intervention, and/or to assess the effectiveness of the treatment. Such intravascular assessment techniques may be performed by medical instruments, e.g., guidewires and/or catheters, disposed within the vasculature of a patient. The vasculature may be tortuous and may include occlusions. Extant devices may be suitable for one type of anatomy, e.g., tortuous vasculature, while being unsuitable for other types of anatomy, e.g., occluded vasculature. Physicians may be obliged to use multiple devices, each with different characteristics, to move within the differing types of anatomy thereby prolonging procedures, increasing costs, and reducing efficiency.

### SUMMARY

Aspects of the present disclosure provide an intraluminal device, e.g., a guidewire or catheter, comprising a mechanism for varying the stiffness of the intraluminal device. The intraluminal device may be introduced into a body lumen of a patient, such as a blood vessel of the patient's vasculature, and may be advanced to a diseased area for assessment and/or treatment of the area. While within the vasculature, the intraluminal device may encounter various features such as branching vessels, tortuous vessels, stenoses, obstructions, etc. Different degrees of stiffness may be advantageous for navigating different features. For example, a low degree of stiffness (high flexibility) may be advantageous when the intraluminal device is navigating a tortuous section of the vasculature. On the other hand, a high degree of stiffness (low flexibility) may be advantageous when the intraluminal device is pushing through a section of the vasculature comprising a stenosis. The ability to vary the stiffness of the intraluminal device during the procedure advantageously allows a physician to choose an appropriate stiffness for a given section of the patient's vasculature and to adjust said stiffness throughout a procedure as sections warranting different degrees of stiffness are encountered.

In one embodiment, an intraluminal device is disclosed. The intraluminal device comprises a flexible elongate body comprising a distal portion configured to be positioned within a body lumen of a patient and a functional device disposed at the distal portion of the flexible elongate body, the functional device configured to obtain physiological data or perform a treatment within the body lumen. The intraluminal device further comprises a stiffening member moveably disposed within and along a longitudinal length of the flexible elongate body without extending beyond a distal end of the flexible elongate body, the stiffening member being moveable during an intraluminal procedure to selectively vary a stiffness of the intraluminal device.

In some embodiments, the stiffening member comprises a flexible shaft. In some embodiments, the flexible shaft is more flexible than the flexible elongate body. In some embodiments, the flexible shaft is less flexible than the flexible elongate body. In some embodiments, the intraluminal device comprises a hub coupled to the stiffening member such that actuation of the hub controls proximal and distal movement of the stiffening member. In some embodiments, the flexible elongate body comprises a sidewall comprising an aperture, wherein the aperture is configured to pass the stiffening member. In some embodiments, the stiffening member comprises a braid, and wherein the braid comprises a proximal end coupled to a proximal portion of the flexible elongate body and a distal end coupled to the distal portion of the flexible elongate body. In some embodiments, the stiffening member comprises a braid, and wherein the braid comprises an electroactive polymer such that a stiffness of the braid varies with voltage applied across the braid. In some embodiments, the intraluminal device comprises an indicator representative of a current stiffness of the intraluminal device. In some embodiments, the indicator is located on the stiffening member. In some embodiments, the functional device comprises at least one of a diagnostic tool or a therapeutic tool. In some embodiments, the functional device comprises at least one of a blood pressure sensor, an ultrasound transducer, a morcellation device, or an ablation device. In some embodiments, the flexible elongate member comprises a catheter or guidewire configured to be positioned within a blood vessel of the patient.

In one embodiment, a method is disclosed. The method comprises inserting a distal portion of a flexible elongate body of an intraluminal device into a body lumen of a patient and selectively translating a stiffening member proximally or distally within the flexible elongate body to vary a stiffness of the intraluminal device during a medical procedure, the stiffening member moveably disposed within and along a longitudinal length of the flexible elongate body without extending beyond a distal end of the flexible elongate body. The method further comprises moving the intraluminal device through different segments of the body lumen based on the varying stiffness of the intraluminal device and obtaining physiological data or performing a treatment within the body lumen using a functional device disposed at the distal portion of the flexible elongate body.

In some embodiments, selectively translating the stiffening member comprises adjusting a voltage applied across the stiffening member. In some embodiments, selectively translating the stiffening member comprises adjusting the position of a hub coupled to the stiffening member such that a position of the hub corresponds to a location of the stiffening member within the flexible elongate body.

In one embodiment, an intraluminal device is disclosed. The intraluminal device comprises a flexible elongate body comprising a distal portion configured to be positioned within a body lumen of a patient and a functional device disposed at the distal portion of the flexible elongate body, the functional device configured to obtain physiological data or perform a treatment within the body lumen. The intraluminal device further comprises a reservoir disposed within the flexible elongate body such that filling or emptying the reservoir selectively varies the stiffness of the intraluminal device during a medical procedure to traverse different segments of the body lumen.

In some embodiments, the reservoir comprises a hydraulic reservoir. In some embodiments, the reservoir comprises a pneumatic reservoir. In some embodiments, the intraluminal device comprises an indicator representative of a current fluid level within the reservoir.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1A is a diagrammatic schematic view of an intraluminal system, including an intraluminal device crossing an occlusion in a body lumen, according to some embodiments of the present disclosure.

FIG. 1B is a diagrammatic schematic view of an intraluminal device within a tortuous body lumen according to some embodiments of the present disclosure.

FIGS. 2A and 2B are diagrammatic schematic views of an intraluminal device according to some embodiments of the present disclosure. FIG. 2A shows the intraluminal device in a lower stiffness configuration. FIG. 2B shows the intraluminal device in a higher stiffness configuration.

FIGS. 3A and 3B are diagrammatic schematic views of an intraluminal device according to some embodiments of the present disclosure. FIG. 3A shows the intraluminal device in a lower stiffness configuration. FIG. 3B shows the intraluminal device in a higher stiffness configuration.

FIGS. 4A and 4B are diagrammatic schematic views of an intraluminal device according to some embodiments of the present disclosure. FIG. 4A shows the intraluminal device in a lower stiffness configuration. FIG. 4B shows the intraluminal device in a higher stiffness configuration.

FIGS. 5A and 5B are diagrammatic schematic views of an intraluminal device according to some embodiments of the present disclosure. FIG. 5A shows the intraluminal device in a lower stiffness configuration. FIG. 5B shows the intraluminal device in a higher stiffness configuration.

FIGS. 6A and 6B are diagrammatic schematic views of an intraluminal device according to some embodiments of the present disclosure. FIG. 6A shows the intraluminal device in a lower stiffness configuration. FIG. 6B shows the intraluminal device in a higher stiffness configuration.

FIG. 7 is a flow diagram of a method according to embodiments of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

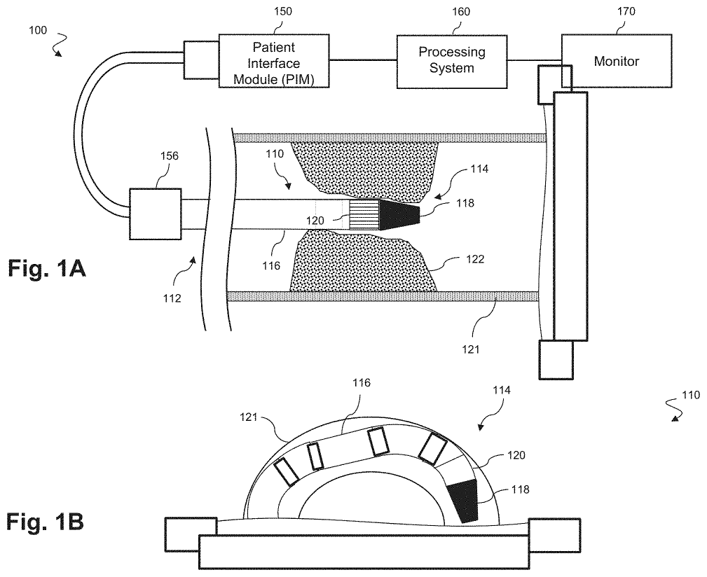

FIG. 1A is a diagrammatic schematic view of an intraluminal system 100 according to some embodiments of the present disclosure. The intraluminal system 100 can include an intraluminal device 110, a patient interface module (PIM) 150, a processing system 160, and/or a monitor 170. The processing system 160 may control the acquisition of medical data and/or the application of a therapy. The processing system 160 may generate an image, e.g., representations of medical data, workflow information, an anatomical image, etc., that is displayed on the monitor 170. The intraluminal device 110 may be structurally arranged (e.g., sized and/or shaped or otherwise configured) to be positioned within anatomy 121 of a patient. In some cases, the anatomy 121 may be a body lumen of the patient and may be a blood vessel of the patient's vasculature. The intraluminal system 100 can be referenced as a medical system, a therapeutic system, and/or a diagnostic system. Similarly, the intraluminal device 110 can be referenced as a medical device, a therapeutic device, and/or a diagnostic device.

The intraluminal device 110 may assess the anatomy 121 and may obtain medical data from within the anatomy 121. For example, the intraluminal device 110 may obtain ultrasound imaging data, e.g., intravascular ultrasound (IVUS) imaging data, may obtain pressure measurements, e.g., blood pressure measurements, may obtain flow data, e.g., blood flow data, may obtain temperature data, or any combination thereof. The intraluminal device 110 may be configured to perform one or more therapeutic procedures within the anatomy 121. For example, the intraluminal device 110 may place a stent, may perform an ablation, may perform an angioplasty, may perform a morcellation, may perform a coiling procedure, may cut through a chronic total occlusion (CTO), or any combination thereof. In that regard, the intraluminal device 110 may comprise any number or combination of therapeutic and/or diagnostic functional devices, such as a transducer or transducer array with a plurality of ultrasound acoustic elements, inflatable balloons, scoring balloons, drug-eluting balloons, drug-coated balloons, cutting balloons, cutting tools, blades, morcellation devices, atherectomy devices, lasers, pressure sensors, e.g., blood pressure sensors, flow sensors, temperature sensors, thermometers, stents, drug-coated stents, needles, ablation devices, ablation electrodes, radiofrequency devices, aspiration devices, and/or other suitable devices, etc.

Generally, the intraluminal device 110 can be a catheter, a guide catheter, or a guide wire. The intraluminal device 110 includes a flexible elongate member 116. As used herein, “elongate member” or “flexible elongate member” includes at least any thin, long, flexible structure structurally arranged (e.g., sized and/or shaped or otherwise configured) to be positioned within a lumen of the anatomy 121. For example, a distal portion 114 of the flexible elongate member 116 may be positioned within the lumen, while a proximal portion 112 of the flexible elongate member 116 may be positioned outside of the body of the patient. The flexible elongate member 116 can include a longitudinal axis LA. In some instances, the longitudinal axis LA can be a central longitudinal axis of the flexible elongate member 116. In some embodiments, the flexible elongate member 116 can include one or more polymer/plastic layers formed of various grades of nylon, Pebax, polymer composites, polyimides, and/or Teflon. In some embodiments, the flexible elongate member 116 can include one or more layers of braided metallic and/or polymer strands. The braided layer(s) can be tightly or loosely braided in any suitable configuration, including any suitable per in count (pic). In some embodiments, the flexible elongate member 116 can include one or more metallic and/or polymer coils. All or a portion of the flexible elongate member 116 may have any suitable geometric cross-sectional profile (e.g., circular, oval, rectangular, square, elliptical, etc.) or non-geometric cross-sectional profile. For example, the flexible elongate member 116 can have a generally cylindrical profile with a circular cross-sectional profile that defines an outer diameter of the flexible elongate member 116. For example, the outer diameter of the flexible elongate member 116 can be any suitable value for positioning within the anatomy 121, including between approximately 1 Fr and approximately 15 Fr, including values such as 3.5 Fr, 5 Fr, 7 Fr, 8.2 Fr, 9 Fr, and/or other suitable values both larger and smaller.

The intraluminal device 110 may or may not include one or more lumens extending along all or a portion of the length of the flexible elongate member 116. If the intraluminal device 110 includes lumen(s), the lumen(s) may be centered or offset with respect to the cross-sectional profile of the intraluminal device 110. During a diagnostic and/or therapeutic procedure, a medical professional typically first inserts the intraluminal device 110 into the lumen of the anatomy 121 and moves the intraluminal device 110 to a desired location within the anatomy 121, such as adjacent to an occlusion 122. In some embodiments, the lumen of the intraluminal device 110 can extend along the entire length of the flexible elongate member 116. As discussed in greater detail below, the lumen of the intraluminal device 110 can be structurally arranged (e.g., sized and/or shaped or otherwise configured) to receive a stiffening member which facilitates the selective varying of the stiffness of the intraluminal device 110.

The anatomy 121 may represent any fluid-filled or surrounded structures, both natural and man-made. For example, the anatomy 121 can be within the body of a patient. Fluid can flow through a lumen of the anatomy 121. The anatomy 121 can be a vessel, such as a blood vessel, in which blood flows. In some instances, the intraluminal device 110 can be referenced as an intravascular device. In various embodiments, the blood vessel may be an artery or a vein of a patient's vascular system, including cardiac vasculature, peripheral vasculature, neural vasculature, renal vasculature, and/or any other suitable anatomy/lumen inside the body. The anatomy 121 can be tortuous in some instances. The intraluminal device 110 may be used to examine any number of anatomical locations and tissue types, including without limitation, organs including the liver, heart, kidneys, gall bladder, pancreas, lungs, esophagus; ducts; intestines; nervous system structures including the brain, dural sac, spinal cord and peripheral nerves; the urinary tract; as well as valves within the vasculature, chambers or other parts of the heart, and/or other systems of the body. In addition to natural structures, the intraluminal device 110 may be used to examine man-made structures such as, but without limitation, heart valves, stents, shunts, filters and other devices.

The occlusion 122 of the anatomy 121 is generally representative of any blockage or other structural arrangement that results in a restriction to the flow of fluid through the lumen of the anatomy 121, for example, in a manner that is deleterious to the health of the patient. For example, the occlusion 122 narrows the lumen such that the cross-sectional area of the lumen and/or the available space for fluid to flow through the lumen is decreased. Where the anatomy 121 is a blood vessel, the occlusion 122 may be a result of plaque buildup, including without limitation plaque components such as fibrous, fibro-lipidic (fibro fatty), necrotic core, calcified (dense calcium), blood, fresh thrombus, and/or mature thrombus. In some instances, the occlusion 122 can be referenced as thrombus, a stenosis, and/or a lesion. Generally, the composition of the occlusion 122 will depend on the type of anatomy being evaluated. Healthier portions of the anatomy 121 may have a uniform or symmetrical profile (e.g., a cylindrical profile with a circular cross-sectional profile). The occlusion 122 may not have a uniform or symmetrical profile. Accordingly, diseased portions of the anatomy 121, with the occlusion 122, will have a non-symmetric and/or otherwise irregular profile. While the anatomy 121 is illustrated in FIG. 1A as having a single occlusion 122, it is understood that the devices, systems, and methods described herein have similar application for anatomy having multiple occlusions.

The intraluminal device 110 may include one or more functional devices 120. For example, the functional device 120 can be a therapeutic tool and/or a diagnostic tool. While one functional device 120 is shown in FIG. 1A, it understood that the intraluminal device 110 can include any suitable number of therapeutic tools and/or diagnostic tools, include two, three, four, or more. In some embodiments, the intraluminal device 110 does not include any therapeutic and/or diagnostic tools. The functional device 120 located at the distal portion 114 of the intraluminal device 110. In some examples, the functional device 120 is a diagnostic component or a sensor, such as a temperature sensor, a thermometer, one or more ultrasound acoustic elements, e.g., an ultrasound transducer or transducer array, a pressure sensor, a flow sensor, or any combination thereof. In that regard, the sensor may in some cases be configured to sense a change in temperature, a change in pressure, a change in flow, emit and/or receive acoustic energy, or any combination thereof. In some cases, the intraluminal device 110 may comprise a plurality of sensors. The plurality of sensors may comprise a plurality of sensors of the same type or may comprise a mixture of types of sensors. For example, the intraluminal device 110 may comprise multiple sensors configured to sense a change in pressure. In another example, the intraluminal device 110 may comprise multiple sensors where some sensors are configured to sense a change in pressure while other sensors are configured to emit and/or receive acoustic energy.

When the sensor is configured to emit and/or receive acoustic energy, the sensor may comprise one or more ultrasound acoustic elements. The acoustic elements may be configured to emit ultrasonic energy into the anatomy 121 while the intraluminal device 110 is positioned within the lumen. In that regard, in some embodiments, the sensor may include ultrasound transducer(s) or transducer arrays(s) and can be configured to generate and emit ultrasound energy into the anatomy 121 in response to being activated by an electrical signal. In some embodiments, the sensor may include a single ultrasound transducer. In some embodiments, the sensor may include an ultrasound transducer array including more than one ultrasound transducer. For example, an ultrasound transducer array can include any suitable number of individual transducers between 2 acoustic elements and 1000 acoustic elements, including values such as 2 acoustic elements, 4 acoustic elements, 36 acoustic elements, 64 acoustic elements, 128 acoustic elements, 500 acoustic elements, 812 acoustic elements, and/or other values both larger and smaller. The acoustic element(s) of the ultrasound transducer can be a piezoelectric micromachined ultrasound transducer (PMUT), capacitive micromachined ultrasonic transducer (CMUT), single crystal, lead zirconate titanate (PZT), PZT composite, other suitable transducer type, and/or combinations thereof. Depending on the transducer material, the manufacturing process for ultrasound transducer(s) can include dicing, kerfing, grinding, sputtering, wafer technologies (e.g., SMA, sacrificial layer deposition), other suitable processes, and/or combinations thereof. The ultrasound transducer array can be any suitable configuration, such as phased array including a planar array, a curved array, a circumferential array, an annular array, etc. For example, the ultrasound transducer array can be a one-dimensional array or a two-dimensional array in some instances. In some instances, the intraluminal device 110 can be a rotational ultrasound device. In some embodiments, the sensor is configured to obtain ultrasound imaging data associated with the anatomy 121, such as the occlusion 122. The ultrasound imaging data obtained by the sensor can be used by a medical professional to diagnose the patient, including evaluating the occlusion 122 of the anatomy 121. In various embodiments, the structure 120 can obtain imaging data associated with intravascular ultrasound (IVUS) imaging, forward looking intravascular ultrasound (FL-IVUS) imaging, intravascular photoacoustic (IVPA) imaging, intracardiac echocardiography (ICE), transesophageal echocardiography (TEE), and/or other suitable imaging modalities.

In some embodiments, the functional device 120 of the intraluminal device 110 may be a treatment component. For example, the treatment component can include a balloon, a stent, a needle, an ablation electrode, mechanical cutting component, a rotational cutting device, an aspiration device, and/or other suitable devices. The treatment component can be a targeted drug delivery device, a drug coated balloon, a drug coated stent, and/or other suitable device configured to deliver a pharmacological agent to the anatomy 121, such as the occlusion 122. For example, the pharmacological agent can be delivered to the anatomy 121 by the treatment component. The treatment component can be positioned at the distal portion 114 of the flexible elongate member 116. When the intraluminal device 110 includes two or more functional devices 120, the sensor(s) and/or treatment component(s) are positioned at the distal portion 114 of the flexible elongate member 116. The relative positioning of the sensor and treatment component can vary in different embodiments. In some cases, one or both of the sensor and the treatment component may be disposed at a distal tip 118 of the intraluminal device 110.

The functional device 120 can be in communication with one or more electrical conductors extending along the length of the flexible elongate member 116. The electrical conductor(s) are may be electrically and/or mechanically coupled to the functional device 120 at the distal portion 114, and an interface 156 at the proximal portion 112. The electrical conductors carry electrical signals between the processing system 160 and the functional device 120. For example, activation and/or control signals can be transmitted from the processing system 160 to the functional device 120 via the electrical conductors. Electrical signals representative of ultrasonic echoes, intraluminal pressure, intraluminal temperature, intraluminal flow, etc., can be transmitted from the functional device 120 to the processing system 160 via the electrical conductors. When the intraluminal device 110 includes two or more functional devices 120, the same electrical conductors can be used for communication between the processing system 160 and sensor(s) and/or treatment component(s), in some embodiments. In other embodiments, different electrical conductors of the intraluminal device 110 can be used for communication between the processing system 160 and sensor(s), and between the processing system 160 and treatment component(s).

The intraluminal device 110 includes an interface 156 at the proximal portion 112 of the flexible elongate member 116. In some embodiments, the interface 156 can include a handle. For example, handle can include one or more actuation mechanisms to control movement of the intraluminal device 110, such as deflection of the distal portion 114. In some embodiments, the interface 156 can include a telescoping mechanism that allows for pullback of the intraluminal device 110 through the lumen. In some embodiments, the interface 156 can include a rotation mechanism to rotate one or more components of the intraluminal device 110 (e.g., the flexible elongate member 116, the sensor, and/or the treatment component). In some embodiments, the interface 156 includes a user interface component (e.g., one or more buttons, a switch, etc.). The processing system 160, the PIM 150, and/or the intravascular device 110 (e.g., the interface 156, sensor, etc.) can include one or more controllers. The controllers can be integrated circuits, such as application specific integrated circuits (ASIC), in some embodiments.

In some embodiments, the PIM 150 performs preliminary processing of the medical data, e.g., ultrasound data, temperature data, flow data, pressure data, etc., prior to relaying the medical data to the processing system 160. In examples of such embodiments, the PIM 150 may perform amplification, filtering, and/or aggregating of the data. In an embodiment, the PIM 150 also supplies high- and low-voltage DC power to support operation of the intraluminal device 110 including circuitry associated with the sensor and/or treatment component. The PIM 150 can be an isolation device as, in various surgical settings, patient safety requirements mandate physical and electrical isolation of the patient from one or more high voltage components.

The processing system 160 may receive medical data (e.g., electrical signals representative of medical data) from the sensor by way of the PIM 150. The processing system 160 can include processing circuit, such as processor and/or memory. In general, the PIM 150 and processing system 160 can include one or more processors, memory, electronic circuitry, hardware, and/or software configured to carry out the functions described herein. The processing system 160 may processes the medical data to reconstruct an image of the anatomy, calculate a fractional flow reserve (FFR) pressure ratio, calculate an instant wave-free ratio (iFR), calculate changes in values, etc. The processing system 160 may output processed medical data for display on the monitor 170. For example, the processing system 160 may output image data, such that an image of the anatomy 121, e.g., a cross-sectional IVUS image of a vessel, to be displayed on the monitor 170. For further example, the processing system 160 may output an FFR and/or iFR for display on the monitor 170. The processing system 160 and/or the monitor 170 can include one or more user interface elements (e.g., touchscreen, keyboard, mouse, virtual buttons on a graphical user interface, physical buttons, etc.) to allow a medical professional to control the intraluminal device 110, including one or more parameters of the sensor and/or treatment component.

As described in greater detail below, the intraluminal device 110 may be structurally arranged or otherwise configured such that a stiffness of the intraluminal device 110 can be varied, e.g., varied during a medical procedure. In some cases, the stiffness of the intraluminal device 110 may be referred to as the rigidity or the flexibility of the intraluminal device 110 or as the resistance-to-bend of intraluminal device 110. Stiffness and flexibility may be inversely related such that high stiffness corresponds to low flexibility. For the sake of clarity, and without limiting the scope of the disclosure, the term stiffness will be the primary term used herein.

Different degrees of stiffness may be advantageous for navigating different features within anatomy 121. For example, a high degree of stiffness may be advantageous when cutting through or pushing past an intravascular obstruction such as occlusion 122 shown in FIG. 1A. On the other hand, a low degree of stiffness may be advantageous when navigating a tortuous section of the vasculature as shown in FIG. 1B. The ability to vary the stiffness of the intraluminal device 110 advantageously allows a physician to choose an appropriate stiffness for a given section of the patient's vasculature and to adjust said stiffness throughout a procedure as sections warranting different degrees of stiffness are encountered.

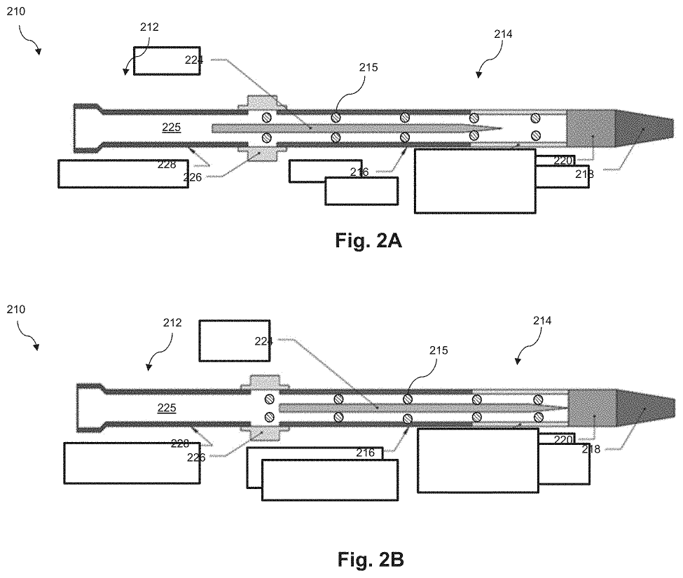

FIGS. 2A and 2B are diagrammatic, schematic views of an intraluminal device 210. The intraluminal device 210 may comprise a proximal portion 212 and a distal portion 214. The intraluminal device 210 may further comprise a sensor 220 and a distal tip 218 disposed at the distal portion 214. A connector assembly 228 may be disposed at the proximal portion 212. The intraluminal device 210 may further comprise a flexible elongate member 216 having a lumen 225 extending within and along the length thereof.

A stiffening member 224 may be disposed within the lumen 225. Though only a single stiffening member 224 is shown, the intraluminal device 210 may comprise a plurality of stiffening members 224, e.g., 2, 3, 4, 5, 6, 7, 8, 9, 10 stiffening members 224. The stiffening member 224 may comprise one or more polymer/plastic layers formed of various grades of nylon, Pebax, polymer composites, polyimides, Teflon, a metal alloy, a pure metal, an electroactive polymer, or any combination thereof. In some cases, the stiffening member 224 may have a higher stiffness than the flexible elongate member 216. In other cases, the stiffening member 224 may have a lower stiffness than the flexible elongate member 216. The stiffening member 224 may comprise a flexible elongate shaft. The stiffening member 224 may be structurally arranged or otherwise configured to provide support to the flexible elongate member 216. In any case, the stiffening member 224 may increase the column strength of the portions of the intraluminal device 210 where it is present. When the stiffening member 224 comprises an electroactive polymer, the position and/stiffness of the stiffening member 224 may be adjusted by adjusting a voltage applied to the stiffening member 224, e.g., a voltage supplied via the conductors described hereinabove.

The stiffening member 224 may be translatable or otherwise moveable within the lumen 225. In that regard, the stiffening member 224 may in some cases be referenced as a translating member, sliding member, moveable member, etc. The stiffening member 224 may be moved proximally and distally within the lumen 225 such that the position of the stiffening member 224 within the lumen 225 may determine the stiffness of the intraluminal device 210 and/or portions of the intraluminal device 210. For example, portions of the intraluminal device 210 containing the stiffening member 224 may be stiffer than portions lacking the stiffening member 224. FIG. 2A illustrates the stiffening member 224 as having been partially retracted as compared with FIG. 2B. Accordingly, the distal portion 214 of the intraluminal device 210 may have a higher stiffness when the stiffening member 224 is in the position shown in FIG. 2B than it has when the stiffening member 224 is in the position shown in FIG. 2A.

When the intraluminal device 210 comprises a plurality of stiffening members 224, e.g., disposed circumferentially within the lumen 225, a physician may choose which stiffening member(s) 224 to extend distally or retract proximally in order to adjust the stiffness of the intraluminal device 210 or a portion of the intraluminal device 210 on a given side. For example, the stiffening member(s) 224 on the right side of the intraluminal device 210 may be fully extended distally while the stiffening member(s) 224 on the left side of the intraluminal device 210 are fully retracted proximally thereby rendering the right side stiffer than the left side. The ability to selectively render one side of the intraluminal device 210 stiffer than the other may facilitate navigating a tortuous vasculature, e.g., by allowing a physician to weaken one side of the intraluminal device 210 in order to increase the tendency of the intraluminal device 210 to bend in that direction, which may facilitate entering a branching vessel.

The position of the stiffening member 224 may be adjusted via a hub 226. In some cases, the hub 226 may be disposed at the proximal portion 212. The hub 226 may be a component of an interface, such as interface 156 described above, and/or may be part of the flexible elongate member 216. The hub 226 may be operated manually and/or may be operated electronically, e.g., by receiving commands from a PIM or medical processing system, such as PIM 150 and processing system 160 discussed above. In some cases, the hub 226 may be operated via one or more mechanical actuators or drive motors.

The stiffening member 224 may be connected to the hub 226 such that movement of the hub 226 effects movement of the stiffening member 224. Movement of the hub 226 may in some cases be referenced as adjustment of the hub 226 and/or as actuation of the hub 226. The connection may be direct, e.g., where the stiffening member 224 is coupled to the hub 226 itself or indirect, e.g., where the stiffening member 224 is coupled to an intermediate element which transfers motion from the hub to the stiffening member 224. Movement of the hub 226 may effect a movement of equal magnitude in the stiffening member 224 or a movement of greater or lesser magnitude as the case may be. A single hub 226 may control a single stiffening member 224. Thus, the intraluminal device 210 may comprise at least as many hubs 226 as stiffening members 224. In other cases, a single hub 226 may control multiple stiffening members 224. The hub 226 may comprise a locking mechanism configured to fix the position of the stiffening member 224.

In some cases, the hub 226 may be structurally arranged or otherwise configured to slide proximally and distally. Sliding the hub 226 in a given direction may move the stiffening member 224 in the same direction or in the opposite direction. In some cases, the hub 226 may be structurally arranged or otherwise configured to rotate, e.g., rotate about a longitudinal axis of the intraluminal device 210, about a pitch axis of the intraluminal device 210, about a yaw axis of the intraluminal device 210, or about any combination of said axes. In such instances, the hub 226 may comprise a reel, spool, ratchet, bobbin, capstan, or any combination thereof. Rotation of the hub 226 may advance and retract the stiffening member 224. For example, rotation of the hub 226 in a first direction may advance (move distally) the stiffening member 224 while rotation in a second direction may retract (move proximally) the stiffening member 224. In some cases, sliding the hub 226 may effect a coarse adjustment of the position of the stiffening member 224 while rotation of the hub 226 may effect a fine adjustment of the position of the stiffening member 224.

The stiffening member 224 may be free floating within the lumen 225 in some instances. Allowing the stiffening member 224 to float freely may advantageously allow the overall size of the intraluminal device 210 to be minimized. Smaller intraluminal devices 210 may be advantageous for navigating narrow passages. In other instances, the stiffening member 224 may be supported by a guide 215. The guide 215 may advantageously ensure that the stiffening member 224 is able to move freely within the lumen 225, e.g., without encountering an obstruction. The guide 215 may comprise one or more rollers, one or more lumens housing the stiffening member 224, one or more tracks, one or more grooves, one or more rings, or any combination thereof. The guide 215 may comprise a lubricious coating to facilitate movement of the stiffening member 224. The guide 215 may facilitate reinforcement of the flexible elongate member 216 by the stiffening member 224 by acting as a force transmitting interface between the two. The guide 215 may extend along the entire length of the flexible elongate member 216 or along a portion less than the entire length of the flexible elongate member 216.

As illustrated in FIGS. 2A and 2B, the guide 215 may comprise a plurality of rollers. The rollers may be spaced at regular intervals or may be placed side-by-side. The rollers may be passive, e.g., may roll in response to force from the stiffening member 224, or may be active, e.g., may be driven by hub 226 and/or one or more actuators/drive motors. In some cases, one subset of rollers includes passive rollers while another subset of rollers includes active rollers. For example, rollers connected to the hub 226 may be active while other rollers are passive. In some cases, active rollers proximate the hub 226 may feed the stiffening member 224 into a track, groove, rings, etc.

The stiffening member 224 may be structurally arranged or otherwise configured to facilitate its movement through guide 215. As illustrated in FIGS. 2A and 2B, the stiffening member 224 may comprise a tapered tip. The tapered tip may help the stiffening member 224 stay within guide 215. For example, when the guide 215 includes rollers, rings, etc., that are spaced apart, the stiffening member 224 may have a tendency to dip slightly as it moves between such features. The tapered tip advantageously reduces the likelihood that the stiffening member 224 will get hung up or stuck on one of the features of the guide 215. The slope of the taper may further facilitate the stiffening member 224 regaining its true course through the guide following such a dip. In some cases, the tip of the stiffening member 224 may be tapered on one side only, e.g., the side in the direction of the expected dip. In some cases, the intraluminal device 210 may include a slot at its distal end into which the stiffening member 224 can slide when fully extended distally. Mating the stiffening member 224 with the slot may advantageously add additional stiffness to the intraluminal device 210, and the taper may assist in such mating, e.g., by effectively increasing the target window for successful mating.

The intraluminal device 210 may comprise an indicator configured to indicate a current stiffness of the intraluminal device 210. The indicator may indicate a current position of the stiffening member 224, e.g., as a percent of extension or retraction, as a length of extension or retraction, etc. In some cases, the indicator may comprise a color gradient or number scale slidable along with the stiffening member 224. In some instances, the indicator may be integrated in the stiffening member 224, such as markings along the length of the stiffening member. The indicator may be connected either directly or indirectly to the hub 226. The indicator may be disposed on the stiffening member 224 in some cases. In that regard, the intraluminal device 210 may feature a transparent viewing window to allow a physician to view the stiffening member 224 within the lumen 225. When the stiffening member 224 comprises an electroactive polymer, the indicator may indicate a voltage applied to the stiffening member 224.

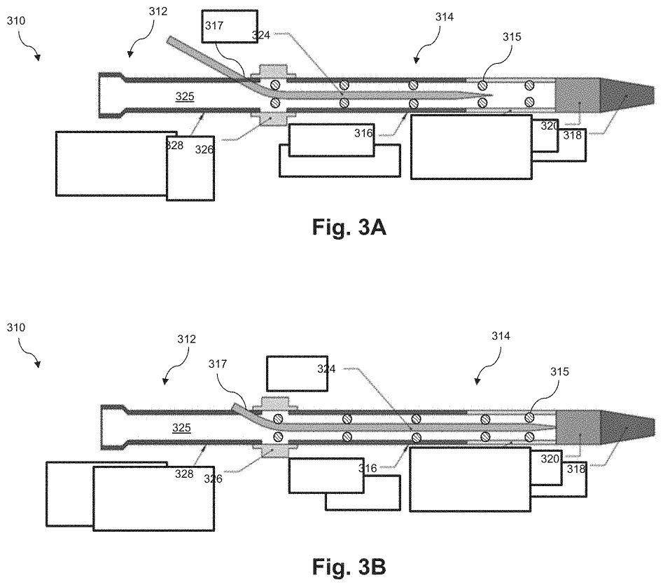

FIGS. 3A and 3B are diagrammatic, schematic views of an intraluminal device 310. The intraluminal device 310 may comprise a proximal portion 312 and a distal portion 314. The intraluminal device 310 may further comprise a sensor 320 and a distal tip 318 disposed at the distal portion 314. A connector assembly 328 may be disposed at the proximal portion 312. The intraluminal device 310 may further comprise a flexible elongate member 316 having a lumen 325 extending within and along the length thereof. A stiffening member 324 may be disposed within the lumen 325 and may be supported by guide 315. The position of the stiffening member 324 may be adjusted by hub 326.

The intraluminal device 310 may comprise an aperture 317 through which the stiffening member 324 is passed. The aperture 317 may be located in the proximal portion 312 of the intraluminal device 310 proximal to the hub 326 and may be disposed in a sidewall of the connector assembly 328 and/or in a sidewall of the flexible elongate member 316. The aperture 317 may comprise a lubricious coating to reduce friction with the stiffening member 324. In some cases, the aperture 317 may comprise a clamp or locking mechanism structurally arranged or otherwise configured such that engagement of the claim or locking mechanism fixes the position of the stiffening member 324. The aperture 317 may permit the stiffening member 324 to be adjusted by hand in addition to or as an alternative to adjustment by hub 326.

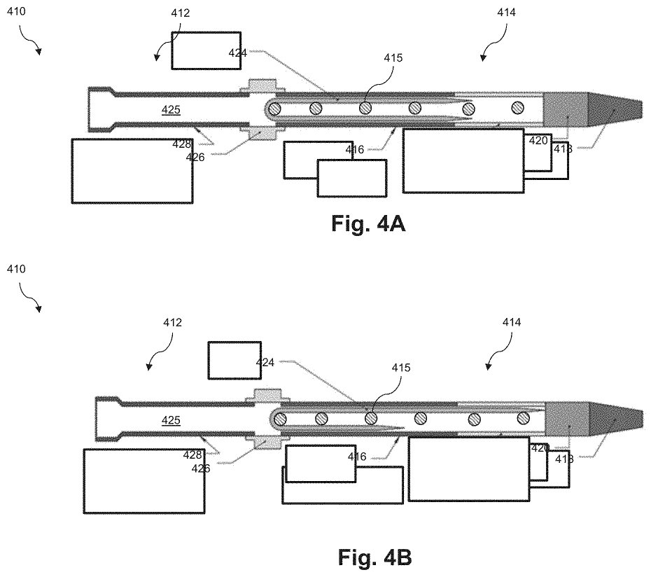

FIGS. 4A and 4B are diagrammatic, schematic views of an intraluminal device 410. The intraluminal device 410 may comprise a proximal portion 412 and a distal portion 414. The intraluminal device 410 may further comprise a sensor 420 and a distal tip 418 disposed at the distal portion 414. A connector assembly 428 may be disposed at the proximal portion 412. The intraluminal device 410 may further comprise a flexible elongate member 416 having a lumen 425 extending within and along the length thereof. A stiffening member 424 may be disposed within the lumen 425 and may be supported by guide 415. The position of the stiffening member 424 may be adjusted by hub 426.

The stiffening member 424 may be folded or doubled back on itself within the hub 426. Though only one folded stiffening member 424 is illustrated in FIGS. 4A and 4B, a plurality of stiffening members 424 may be folded. Folding the stiffening member 424 may provide additional stiffness above what would be provided if the stiffening member 424 were unfolded. The length of the stiffening member 424 may be such that when the stiffening member 424 is folded exactly in half within the lumen 425, the ends of the stiffening member 425 are positioned at some distance proximal to a distal end of the flexible elongate member 416. The length of the stiffening member 424 may be chosen such some desired portion of the stiffening member 424 remains doubled when the stiffening member 424 is fully extended distally, e.g., as shown in FIG. 4B.

The portion of the stiffening member 424 that remains doubled may provide more stiffness than the portion of the stiffening member 424 extending beyond the doubled portion. Accordingly, the stiffening member 425 may advantageously provide a gradient of stiffness along the length of the intraluminal device 410, said gradient of stiffness being adjustable by adjusting the position of the stiffening member 424. When the stiffening member 424 is folded, extension of one end of the stiffening member 424 distally may result in the retraction of the other end proximally. The side of the intraluminal device 410 on which the stiffening member 424 is extended may be stiffer than the side on which the stiffening member 424 is retracted. Thus, a physician may be able to adjust the stiffness of individual sides of the intraluminal device 410, which confers the advantages described hereinabove.

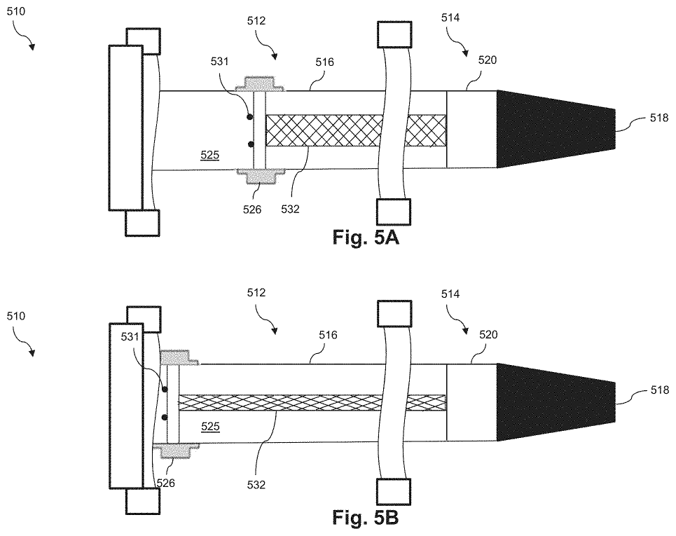

FIGS. 5A and 5B are diagrammatic, schematic views of an intraluminal device 510. The intraluminal device 510 may comprise a proximal portion 512 and a distal portion 514. The intraluminal device 510 may further comprise a sensor 520 and a distal tip 518 disposed at the distal portion 514. The intraluminal device 510 may further comprise a flexible elongate member 516 having a lumen 525 extending within and along the length thereof. A stiffening member in form of braid 532 may be disposed within the lumen 525. Though only a single braid 532 is shown in FIGS. 5A and 5B, a plurality of braids 532 may be used in some instances. Though not illustrated in FIGS. 5A and 5B, the braid 532 may be supported by a guide as similarly described hereinabove.

The braid 532 may comprise one or more polymer/plastic layers formed of various grades of nylon, Pebax, polymer composites, polyimides, Teflon, a metal alloy, a pure metal, an electroactive polymer, or any combination thereof. The braid 532 may be connected at its distal end to a connection point, e.g., a ring, mount, hook, clamp, etc., at the distal portion 514. The braid 532 may be connected at its proximal end to hub 526. In that regard, strands of the braid 532 may pass through a shaft of the hub 526 and may be attached thereto by knots 531. Though only two knots 531 are illustrated in FIGS. 5A and 5B, any number of knots 531 may be used, including an equal number of knots 531 to the number strands of the braid 532. The hub 526 may be slidable and/or rotatable where movement of the hub 526 effects movement of the braid 532.

The stiffness of the intraluminal device 510 may be adjusted by adjusting tension on the braid 532. Tension may be applied or released by adjustment of the hub 526. For example, FIG. 5A illustrates the braid 532 in an un-tensioned state while FIG. 5B illustrates the braid 532 in a tensioned state, the hub 526 in FIG. 5B having been slid proximally from its position in FIG. 5A. Increased tension on the braid 532 may cause increased stiffness of the intraluminal device 510. In some cases, sliding the hub 526 may effect a coarse adjustment of the tension on the braid 532 while rotation of the hub 526 may effect a fine adjustment of the tension on the braid 532. When the braid 532 comprises an electroactive polymer, the tension on the braid 532 may be adjusted by adjusting a voltage applied to the braid 532, e.g., a voltage supplied via the conductors described hereinabove.

When the intraluminal device 510 comprises a plurality of braids 532, e.g., disposed circumferentially within the lumen 525, a physician may choose which braids 524 to tension in order to adjust the stiffness of the intraluminal device 510 or a portion of the intraluminal device 510 on a given side. For example, braids 532 on the right side of the intraluminal device 510 may be tensioned while the braids 532 on the left side of the intraluminal device 510 are slacked thereby rendering the right side stiffer than the left side. Thus, a physician may be able to adjust the stiffness of individual sides of the intraluminal device 510, which confers the advantages described hereinabove.

The intraluminal device 510 may comprise an indicator configured to indicate a current stiffness of the intraluminal device 510. The indicator may indicate a tension on the braid 532. In some cases, the indicator may comprise a color gradient or number scale slidable along with the hub 526. The indicator may be connected either directly or indirectly to the hub 526. The indicator may be disposed on the braid 532 in some cases. In that regard, the intraluminal device 510 may feature a transparent viewing window to allow a physician to view the braid 532 within the lumen 525. When the braid 532 comprises an electroactive polymer, the indicator may indicate a voltage applied to the braid 532.

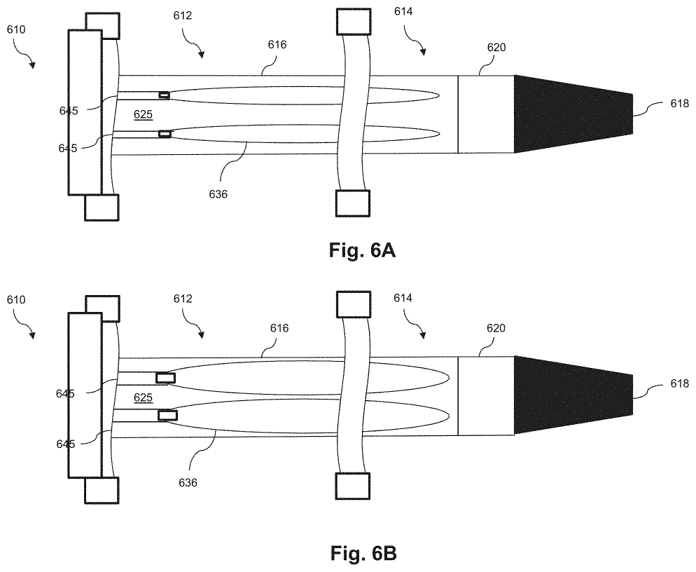

FIGS. 6A and 6B are diagrammatic, schematic views of an intraluminal device 610. The intraluminal device 610 may comprise a proximal portion 612 and a distal portion 614. The intraluminal device 610 may further comprise a sensor 620 and a distal tip 618 disposed at the distal portion 614. The intraluminal device 610 may further comprise a flexible elongate member 616 having a lumen 625 extending within and along the length thereof. The intraluminal device 610 comprises one or more stiffening members in the form of reservoirs 636 disposed within the lumen 625.

The reservoirs 636 may be structurally arranged or otherwise configured to receive a fluid, e.g., water or saline, and/or a gas, e.g., air. In that regard, the reservoir 636 may comprise a hydraulic reservoir and/or a pneumatic reservoir as the case may be. Fluid and/or gas may be delivered into the reservoirs 636 via conduits 645 which may extend within the lumen 625 to a position in the proximal portion 612, e.g., a position outside the body of a patient. The fluid and/or gas chosen to fill reservoirs 636 may be selected to minimize risk to a patient in the event of a rupture of the reservoirs 636. Reservoirs 636 may be supported within the lumen 625 by a guide, such as those described hereinabove. The reservoirs 636 can include one or more polymer/plastic layers formed of various grades of nylon, Pebax, polymer composites, polyimides, and/or Teflon. In that regard, the reservoirs 636 may be made of a flexible material and may have a low stiffness in an unfilled state. In some cases, the reservoirs 636 may expand as they are filled. The stiffness of the intraluminal device 610 may be increased by filling the reservoirs 636. Stiffness of the intraluminal device 610 may be decreased by emptying the reservoirs 636.

When the intraluminal device 610 comprises a plurality of reservoirs 636, e.g., disposed circumferentially within the lumen 625, a physician may choose which reservoirs 636 to fill in order to adjust the stiffness of the intraluminal device 610 or a portion of the intraluminal device 610 on a given side. For example, reservoirs 636 on the right side of the intraluminal device 610 may be filled while the reservoirs 636 on the left side of the intraluminal device 610 remain empty thereby rendering the right side stiffer than the left side. Thus, a physician may be able to adjust the stiffness of individual sides of the intraluminal device 610, which confers the advantages described hereinabove.

The intraluminal device 610 may comprise an indicator configured to indicate a current stiffness of the intraluminal device 610. The indicator may indicate a current fluid level or fill level of the reservoirs 636, a pressure within the reservoirs 636, or any combination thereof. In some cases, the indicator may comprise a color gradient or number scale. The indicator may be connected either directly or indirectly to a hub controlling the flow of fluid and/or gas into the reservoirs 636.

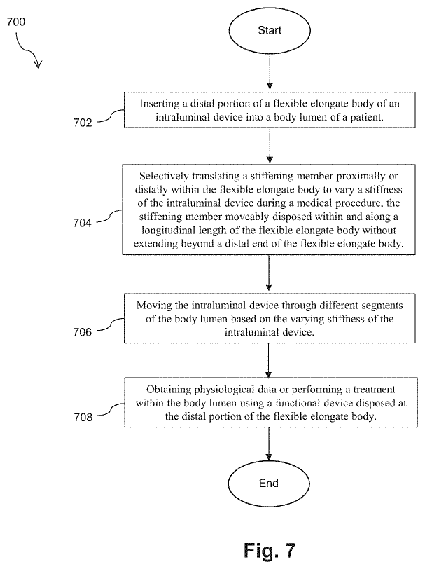

FIG. 7 is a flow chart of a method 700. Portions of the method 700 may correspond to techniques discussed hereinabove. The method begins at block 702 where a distal portion of a flexible elongate body of an intraluminal device is inserted into a body lumen of a patient. At block 704, a stiffening member is selectively translated proximally or distally within the flexible elongate body to vary a stiffness of the intraluminal device during a medical procedure, the stiffening member moveably disposed within and along a longitudinal length of the flexible elongate body without extending beyond a distal end of the flexible elongate body. In some cases, selectively translating the stiffening member may comprise adjusting a voltage applied across the stiffening member. In some cases, selectively translating the stiffening member comprises adjusting the position of a hub coupled to the stiffening member such that a position of the hub corresponds to a location of the stiffening member within the flexible elongate body. The intraluminal device is moved, at block 706, through different segments of the body lumen based on the varying stiffness of the intraluminal device. The method continues at block 708 where physiological data is obtained or a treatment is performed within the body lumen using a functional device disposed at the distal portion of the flexible elongate body.

The systems, devices, and methods of the present disclosure can include features described in U.S. Provisional App. No. 62/548,881 (Atty. Dkt. No. 2017PF02317/44755.1813PV01), titled “Adjustable Flexibility/Stiffness Intraluminal Device and Associated Devices, Systems, and Methods,” and filed on Aug. 22, 2017, the entirety of which is hereby incorporated by reference herein.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intraluminal device, comprising:
a flexible elongate body comprising a distal portion configured to be positioned within a body lumen of a patient;
a functional device disposed at the distal portion of the flexible elongate body, the functional device configured to obtain physiological data or perform a treatment within the body lumen; and
a stiffening member moveably disposed within and along a longitudinal length of the flexible elongate body without extending beyond a distal end of the flexible elongate body, the stiffening member being moveable during an intraluminal procedure to selectively vary a stiffness of the intraluminal device.

2. The intraluminal device of claim 1, wherein the stiffening member comprises a flexible shaft.

3. The intraluminal device of claim 2, wherein the flexible shaft is more flexible than the flexible elongate body.

4. The intraluminal device of claim 2, wherein the flexible shaft is less flexible than the flexible elongate body.

5. The intraluminal device of claim 1, further comprising a hub coupled to the stiffening member such that actuation of the hub controls proximal and distal movement of the stiffening member.

6. The intraluminal device of claim 1, wherein the flexible elongate body comprises a sidewall comprising an aperture, wherein the aperture is configured to pass the stiffening member.

7. The intraluminal device of claim 1, wherein the stiffening member comprises a braid, and wherein the braid comprises a proximal end coupled to a proximal portion of the flexible elongate body and a distal end coupled to the distal portion of the flexible elongate body.

8. The intraluminal device of claim 1, wherein the stiffening member comprises a braid, and wherein the braid comprises an electroactive polymer such that a stiffness of the braid varies with voltage applied across the braid.

9. The intraluminal device of claim 1, further comprising an indicator representative of a current stiffness of the intraluminal device.

10. The intraluminal device of claim 9, wherein the indicator is located on the stiffening member.

11. The intraluminal device of claim 1, wherein the functional device comprises at least one of a diagnostic tool or a therapeutic tool.

12. The intraluminal device of claim 1, wherein the functional device comprises at least one of a blood pressure sensor, an ultrasound transducer, a morcellation device, or an ablation device.

13. The intraluminal device of claim 1, wherein the flexible elongate member comprises a catheter or guidewire configured to be positioned within a blood vessel of the patient.

14. A method, comprising:
inserting a distal portion of a flexible elongate body of an intraluminal device into a body lumen of a patient;
selectively translating a stiffening member proximally or distally within the flexible elongate body to vary a stiffness of the intraluminal device during a medical procedure, the stiffening member moveably disposed within and along a longitudinal length of the flexible elongate body without extending beyond a distal end of the flexible elongate body;
moving the intraluminal device through different segments of the body lumen based on the varying stiffness of the intraluminal device; and
obtaining physiological data or performing a treatment within the body lumen using a functional device disposed at the distal portion of the flexible elongate body.

15. The method of claim 14, wherein selectively translating the stiffening member comprises adjusting a voltage applied across the stiffening member.

16. The method of claim 14, wherein selectively translating the stiffening member comprises adjusting the position of a hub coupled to the stiffening member such that a position of the hub corresponds to a location of the stiffening member within the flexible elongate body.

17. An intraluminal device, comprising:
a flexible elongate body comprising a distal portion configured to be positioned within a body lumen of a patient;
a functional device disposed at the distal portion of the flexible elongate body, the functional device configured to obtain physiological data or perform a treatment within the body lumen; and
a reservoir disposed within the flexible elongate body such that filling or emptying the reservoir selectively varies the stiffness of the intraluminal device during a medical procedure to traverse different segments of the body lumen.

18. The intraluminal device of claim 17, wherein the reservoir comprises a hydraulic reservoir.

19. The intraluminal device of claim 17, wherein the reservoir comprises a pneumatic reservoir.

20. The intraluminal device of claim 17, further comprising an indicator representative of a current fluid level within the reservoir.
